(19) United States
(12) Patent Application Publication
(10) Pub. No.: US 2011/0223245 A1
(43) Pub. Date: Sep. 15, 2011

(54) CONTROLLED-RELEASE FORMULATIONS OF PRAMIPEXOLE

(75) Inventors: Umit Cifter, Istanbul (TR); Ali Turkyilmaz, Istanbul (TR); Hasan Ali Turp, Istanbul (TR); Gulay Yelken, Istanbul (TR); Levent Oner, Ankara (TR)

(73) Assignee: Sanovel IIac Sanayi Ve Ticaret Anonim Sirketi, Istanbul (TR)

(21) Appl. No.: 13/044,709

(22) Filed: Mar. 10, 2011

(30) Foreign Application Priority Data
Mar. 11, 2010 (TR) 201001862

(51) Int. Cl. A61K 31/428; A61K 9/48; A61P 25/16; A61P 25/00; B29C 43/36

(52) U.S. Cl. 424/452; 514/367; 264/299

(57) ABSTRACT

A controlled-release pharmaceutical formulation, comprising pramipexole or a pharmaceutically acceptable salt of pramipexole, colloidal silicone dioxide, and glyceryl behenate.

### CROSS REFERENCE TO RELATED APPLICATIONS

This application is based upon Turkish Patent Application No. TR201001862, filed Mar. 11, 2010, under relevant sections of 35 USC §119, the entire contents of this application being incorporated by reference herein.

### TECHNICAL FIELD OF THE INVENTION

The present invention relates to a novel pharmaceutical formulation consisting of pramipexole or pharmaceutically acceptable salt or hydrate of pramipexole. The invention more particularly relates to controlled-release formulations of pramipexole, allowing the latter to become dispersed uniformly within a matrix tablet and be used as a single daily dose.

### BACKGROUND OF THE INVENTION

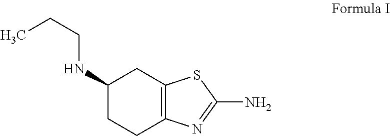

Pramipexole is a non-ergot dopamine agonist. The chemical designation of pramipexole is (S)-2-amino-4,5,6,7-tetrahydro-6-(propylamino)benzothiazole, with the chemical structure as shown in Formula I. Pramipexole has a quite high water solubility.

Immediate-release tablets of pramipexole is commercially available under the trademark Mirapex®, orally administered three times per day, and comprises 0.125 mg, 0.25 mg, 0.5 mg, 1 mg or 1.5 mg pramipexole dihydrochloride monohydrate as the active agent.

Pramipexole, along with its production processes, are disclosed in EP0186087B1 and is particularly known in treating schizophrenia and Parkinson's disease.

The application EP1531814 concerns dispersing an orally deliverable sustained-release tablet composition comprising a water-soluble salt of pramipexole in a matrix. It further comprises a hydrophilic polymer and a starch having a tensile strength of at least about 0.15 kNcm².

The application EP1536792 concerns an oral formulation of pramipexole, which, on average, and in a single dose per day in vitro release profile, does not dissolve more than 20% within 2 hours, and of which the in vivo absorption is more than 20% within 2 hours and/or more than 40% within 4 hours.

In the application EP2135602, in turn, is disclosed an extended-release tablet formulation of pramipexole comprising at least one swelling polymer other than pregelatinized starch in a matrix.

Additionally, the controlled-release formulations disclosed in the patents WO2007054976, US2009004281 comprise coating substances.

Producing controlled-release tablets of pramipexole and similar active agents with high solubility rates and desired release profiles in these tablets is quite difficult. Generating release amounts at desired time intervals bears vital importance with respect to patients.

Furthermore, the amount of active agent included in pramipexole formulations is quite low and this fact makes its production difficult. It is further known that low-dose pharmaceutical compositions are problematic in providing a uniform dispersion in the formulations in which they are present. This is because it is difficult to homogenize the active agent present in the final dosage form in lower amounts and other problems may be encountered while they are compressed. This results in final dosage forms with improper content uniformity.

It is known that uniformly distributing pharmaceutical active agents with pharmaceutical auxiliaries is a desired case in formulating low-dose pharmaceutical active agents for use in patients to be treated for obtaining a proper dosage and homogeneity. Accordingly, it is further desired to provide improved processes for preparing solid oral dosage forms, which have high uniformity and disperse satisfactorily while being administered orally.

In result, there is a need towards pharmaceutical compositions, providing satisfactory distribution of pramipexole or pharmaceutically-acceptable salts, solvates, or hydrates thereof, and showing proper content uniformity and desired release profiles.

### SUMMARY AND DESCRIPTION OF THE INVENTION

The present invention relates to controlled-release formulations of pramipexole, eliminating all aforesaid problems and brining additional advantages to the relevant prior art.

Accordingly, the main object of the present invention is to obtain controlled-release formulations comprising pramipexole, which are stable and have a desired release profile.

Another object of the present invention is to provide a uniform dispersion of pramipexole in the formulation, thereby obtaining a formulation with a proper therapeutic use range.

A further object of the present invention is to provide a uniform dispersion of pramipexole in the formulation, thereby facilitating the production process.

Yet a further object of the present invention is to embody a controlled-release formulation of pramipexole, without requiring to apply any coating thereon.

Accordingly, a pharmaceutical formulation, which does not contain any coating substance has been developed, to achieve all objects referred to above and to emerge from the following detailed description.

According to a preferred embodiment of the present invention, said novelty is carried out with pramipexole or a pharmaceutically acceptable salt thereof, colloidal silicone dioxide, and glyceryl behenate.

According to a preferred embodiment of the present invention, pramipexole or a pharmaceutically acceptable salt of pramipexole is released by 30% at most, preferably by 20% at most in 2 hours; by 35-65% and preferably by 40-65% in 4 hours; and by 85% at least in 16 hours.

According to a preferred embodiment of the present invention, glyceryl behenate is present in an amount of 5 to 90% by weight of the total composition.

According to a preferred embodiment of the present invention, glyceryl behenate is present in an amount of 10 to 70% by weight of the total composition.

According to another preferred embodiment of the present invention, said pramipexole is in the form of dihydrochloride monohydrate.

According to a further preferred embodiment of the present invention, said formulation further comprises at least one or a mixture of xanthan gum, guar gum, a mixture of a locust bean gum-derived heterosaccharide and a dextrose-derived saccharide, or castor oil, hydrogenated castor oil, cellulose acetate phthalate, polyvinyl acetate phthalate, hydroxypropyl methyl cellulose acetate succinate, poly(methylinyl ether/maleic acid) mono ethyl ester, poly(methyl ether/maleic acid) n-butyl ester.

In a preferred embodiment according to the present invention, at least one or a mixture of the following is/are used as a diluent: lactose, microcrystalline cellulose, starch, mannitol, calcium phosphate anhydrate, dibasic calcium phosphate dihydrate, calcium phosphate trihydrate, sugars, sorbitol, mannitol, xylitol, sucrose polysaccharides. Said diluent is preferably microcrystalline cellulose and dibasic calcium phosphate.

In a preferred embodiment according to the present invention, polyvinylprolidone is comprised as a binder.

In a preferred embodiment according to the present invention, magnesium stearate is comprised as a lubricant.

A further preferred embodiment according to the present invention provides a method for preparing a pharmaceutical formulation, this method comprising the steps of:
a) mixing together with colloidal silicone dioxide and pramipexole dihydrochloride monohydrate after sieving;
b) adding dibasic calcium phosphate dihydrate, glyceryl behenate, polymethacrylate, copovidone(polyvinylpyrolidone-vinyl acetate copolymer), microcrystalline cellulose into this powder mixture and mixing the resulting mixture;
c) adding magnesium stearate to the mixture after sieving and mixing the resultant mixture for a short period of time; and
d) compressing the resulting mixture into tablets, or filling this powder mixture into capsules.

Another preferred embodiment according to the present invention provides a method for preparing a pharmaceutical formulation, this method comprising the steps of:
a) mixing together with polyvinylprolidone, dibasic calcium phosphate dihydrate and pramipexole dihydrochloride monohydrate after sieving in a high-shear granulator;
b) melting glyceryl behenate and spraying this melt into the powder mixture prepared, thereby yielding wet granules and then cooling and sieving the wet granules;
c) adding polymethacrylate, microcrystalline cellulose and colloidal silicone dioxide into this powder mixture and mixing the resulting mixture;
d) adding magnesium stearate into this mixture and blending the resulting mixture until a uniform powder mixture is obtained; and
e) compressing the blended mixture in order to obtain tablets, or filling the powder mixture into capsules.

A further preferred embodiment according to the present invention provides a method for preparing a pharmaceutical formulation, this method comprising the steps of:
a) dissolving pramipexole dihydrochloride monohydrate and polyvinylpyrrolidone in water and/or alcohol to produce a pramipexole dihydrochloride monohydrate solution;
b) adding dibasic calcium phosphate dehydrate into the resulting solution while it is mixed, then it is blended in a high-shear granulator to produce wet granules;
c) adding solution of polymethacrylates into the resulting mixture;
d) sieving, and then drying the wet granules;
e) adding glyceryl behenate, microcrystalline cellulose and then colloidal silicone dioxide into the resulting powder;
f) adding magnesium stearate into this mixture and blending the resulting mixture until a uniform powder mixture is obtained; and
g) compressing the blended mixture in order to obtain tablets, or filling the powder mixture into capsules.

Another preferred embodiment according to the present invention provides a method for preparing a pharmaceutical formulation, this method comprising the steps of:
a) dissolving pramipexole dihydrochloride monohydrate and polyvinylpyrrolidone in water and/or alcohol to produce a pramipexole dihydrochloride monohydrate solution;
b) while the resulting solution is mixed, adding dibasic calcium phosphate dihydrate, microcrystalline cellulose, and glyceryl behenate into this solution and mixing the same, thereafter blending it in a high-shear granulator to produce granules;
c) adding solution of polymethacrylates into resulting mixture;
d) sieving, and then drying the wet granules, thereafter disintegrating the dry granules;
e) adding colloidal silicone dioxide and mixing the resultant mixture;
f) adding magnesium stearate into this mixture and blending the resulting mixture until a uniform mixture is obtained; and
g) compressing the blended mixture in order to obtain tablets, or filling the powder mixture into capsules.

A further preferred embodiment according to the present invention provides a method for preparing a pharmaceutical formulation, this method comprising the steps of:
a) dissolving pramipexole dihydrochloride monohydrate and polyvinylpyrrolidone in water and/or alcohol to produce a pramipexole dihydrochloride monohydrate solution;
b) mixing dibasic calcium phosphate dihydrate, microcrystalline cellulose, and glyceryl behenate in a high-shear granulator;
c) spraying the pramipexole dihydrochloride monohydrate solution into a blended powder mixture of dibasic calcium phosphate dihydrate, microcrystalline cellulose, and glyceryl behenate, thereby obtaining wet granules;
d) sieving, and then drying the wet granules, thereafter disintegrating the dry granules;
e) adding colloidal silicone dioxide and mixing the resultant mixture;
f) adding magnesium stearat into this mixture and blending the resulting mixture until a uniform powder mixture is obtained; and
g) compressing the blended mixture in order to obtain tablets, or filling the powder mixture into capsules.

Another preferred embodiment according to the present invention provides a method for preparing a pharmaceutical formulation, this method comprising the steps of:
a) dissolving pramipexole dihydrochloride monohydrate and polyvinylpyrrolidone in water and/or alcohol to produce a pramipexole dihydrochloride monohydrate solution;
b) while the resulting solution is mixed, adding dibasic calcium phosphate dihydrate, microcrystalline cellulose, and glyceryl behenate into this solution and mixing the same, thereafter blending it in a high-shear granulator to produce granules;
c) sieving, and then drying the wet granules, thereafter disintegrating the dry granules;
d) adding colloidal silicone dioxide and mixing the resultant mixture;
e) adding magnesium stearate into this mixture and blending the resulting mixture until a uniform powder mixture is obtained; and
f) compressing the blended mixture in order to obtain tablets, or filling the powder mixture into capsules.

In a further preferred embodiment of the present invention, said pharmaceutical formulation consisting of:
a) pramipexole dihydrochloride monohydrate at 0.05 to 5% by weight;
b) dibasic calcium phosphate dihydrate at 5 to 90% by weight;
c) glyceryl behenate and polymethacrylates at 5 to 90% by weight;
d) polyvinylprolidone at 0.1 to 30% by weight;
e) microcrystalline cellulose at 5 to 90% by weight;
f) silicone dioxide at 0.1 to 10% by weight; and
g) magnesium stearate at 0.1 to 10% by weight.

### DETAILED DESCRIPTION OF THE INVENTION

### Example

| | Amount (%) |
|---|---|
| Content | (w/w) |
| pramipexole dihydrochloride monohydrate | 0.05-5 |
| dibasic calcium phosphate dihydrate | 5-90 |
| glyceryl behenate and polymethacrylates | 5-90 |
| polyvinylprolidone | 0.1-30 |
| microcrystalline cellulose | 5-90 |
| colloidal silicone dioxide | 0.1-10 |
| magnesium stearate | 0.1-10 |

The formulation according to the present invention can be obtained via various methods. In the first method, pramipexole dihydrochloride monohydrate and silicone dioxide are sieved together and mixed. Thus, pramipexole, which is quite low by volume and weight is uniformly mixed with silicone dioxide, and is surrounded by colloidal silicone dioxide particles. This allows to disperse pramipexole uniformly within the formulation. Thereafter glyceryl behenate, polymethacrylates, microcrystalline cellulose dibasic calcium phosphate dihydrate and copovidone(polyvinylpyrolidone-vinyl acetate copolymer) is added into the first mixture and mixed together. Into this mixture formed, sieved magnesium stearate is added and the resulting mixture is mixed again. The final mixture is compressed into tablets, or filled into capsules.

In the granulation method realized via melting, pramipexole dihydrochloride monohydrate, polyvinylprolidone and dibasic calcium phosphate dihydrate are sieved together and mixed. Then glyceryl behenate is melted and this melt obtained is sprayed into the powder mixture, thereby yielding wet granules. Wet granules formed are cooled and sieved. Then, microcrystalline cellulose and polymethacrylates are mixed together with granules and then colloidal silicone dioxide is mixed with resulting powder. Into this mixture formed, sieved magnesium stearate is added and the resulting mixture is mixed again. The final mixture is compressed into tablets, or filled into capsules.

In the method realized via wet granulation, pramipexole dihydrochloride monohydrate and polyvinylpyrrolidone are dissolved in water and/or alcohol to produce a solution of pramipexole dihydrochloride monohydrate. Dibasic calcium phosphate dehydrate is added into the resulting solution, while it is mixed, mixing is continued, and it is then blended in a high-shear granulator to produce wet granules. Thereafter, the granulation step is continued with a solution of polymethacrylates. The final wet granules are sieved and dried. Then glyceryl behenate and microcrystalline cellulose are added into and then colloidal silicone dioxide is mixed with resulting powder. Into this mixture formed, sieved magnesium stearate is added and the resulting mixture is mixed again. The final mixture is compressed into tablets, or filled into capsules.

Water, alcohol or a mixture of water-alcohol is used in the processes as a solvent.

Glyceryl behenate is characterized by not swelling upon contact with water.

Thanks to the present invention developed, a stable controlled-release formulation of pramipexole is surprisingly obtained, by which pramipexole is uniformly dispensed in the formulation and the desired release profile is achieved. The controlled-release pharmaceutical formulation, comprising:
a) pramipexole or a pharmaceutically acceptable salt of pramipexole as an active agent;
b) colloidal silicone dioxide as an excipient;
c) glyceryl behenate as a controlled release agent; and
d) further comprising another controlled release agent which is selected from the group of xanthan gum, guar gum, a mixture of a locust bean gum-derived heterosaccharide and a dextrose-derived saccharide, or castor oil, hydrogenated castor oil, cellulose acetate phthalate, polyvinyl acetate phthalate, hydroxypropyl methyl cellulose acetate succinate, poly(methylinyl ether/maleic acid) mono ethyl ester, poly(methyl ether/maleic acid) n-butyl ester, polymethacrylates and mixture thereof.

First controlled release agent is glyceryl behenate. Another controlled release agent is without glyceryl behenate. The weight ratio of pramipexole to glyceryl behenate is in the range of 0.01-1, this range allowing to produce a release profile desired for controlled release purposes.

It is also possible to use the following additional excipients in the formulation.

Suitable binders include, but are not restricted to, at least one or a mixture of polyvinylprolidone, gelatin, sugars, glucose, natural gums, synthetic celluloses, polymethacrylate, hydroxypropyl methyl cellulose, hydroxypropyl cellulose, carboxymethyl cellulose, methyl cellulose, and other cellulose derivatives.

Suitable glidants include, but are not restricted to, at least one or a mixture of colloidal silicone dioxide, talk, aluminum silicate.

Suitable lubricants include, but are not restricted to, at least one or a mixture of sodium stearil fumarat, magnesium stearat, polyethylene glycol, stearic acid, metal stearates, boric acid, sodium chloride benzoate and acetate, sodium or magnesium lauryl sulfate.

Suitable preservatives include, but are not restricted to, at least one or a mixture of methyl paraben and propyl paraben and salts thereof (e.g. sodium or potassium salts), sodium benzoate, citric acid, benzoik acid, butylated hydroxytoluene and butylated hydroxyanisole.

Suitable colorants include, but are not restricted to, at least one or a mixture of food, drug, and cosmetic (FD&C) dyes (FD&C blue, FD&C green, FD&C red, FD&C yellow, FD&C lake), ponceau, indigo Drug & cosmetic (D&C) blue, indigotine FD&C blue, carmoisine indigotine (indigo Carmine); iron oxides (e.g. iron oxide red, yellow, black), quinoline yellow, flame red, brilliant red (carmine), carmoisine, sunset yellow.

This formulation can be used to treat Parkinson's disease and restless leg syndrome.

The protection scope of the present invention is set forth in the annexed Claims and cannot be restricted to the illustrative disclosures given above, under the detailed description. Any alternative embodiments to be produced by persons skilled in the art according to the basic principles, which are under the protection scope as set forth in the Claims, shall be an infringement of the present invention.

1. A controlled-release pharmaceutical formulation, comprising:
a) pramipexole or a pharmaceutically acceptable salt of pramipexole as an active agent;
b) colloidal silicone dioxide as an excipient;
c) glyceryl behenate as a first controlled release agent; and
d) further comprising a second controlled release agent which is selected from the group of xanthan gum, guar gum, a mixture of a locust bean gum-derived heterosaccharide and a dextrose-derived saccharide, or castor oil, hydrogenated castor oil, cellulose acetate phthalate, polyvinyl acetate phthalate, hydroxypropyl methyl cellulose acetate succinate, poly(methylinyl ether/maleic acid) mono ethyl ester, poly(methyl ether/maleic acid) n-butyl ester, polymethacrylates and mixture thereof.

2. A pharmaceutical formulation according to claim 1, wherein the second controlled release agents are xanthan gum, guar gum, a mixture of a locust bean gum-derived heterosaccharide and a dextrose-derived saccharide, polymethacrylates and mixture thereof.

3. A pharmaceutical formulation according claim 1, wherein the weight ratio of pramipexole to glyceryl behenate is between about 0.01 to 1.

4. A pharmaceutical formulation according to claim 1, wherein the weight ratio of glyceryl behenate is 5 to 90% by weight of the total amount.

5. A pharmaceutical formulation according to claim 1, wherein the weight ratio of glyceryl behenate is 10 to 70% by weight of the total amount.

6. A pharmaceutical formulation according to claim 1, wherein pramipexole is present in the form of pramipexole dihydrochloride monohydrate.

7. A pharmaceutical formulation according to claim 1, wherein the further excipient is used as a diluent and comprises at least one or a mixture of lactose, microcrystalline cellulose, starch, mannitol, calcium phosphate anhydrate, dibasic calcium phosphate dihydrate, calcium phosphate trihydrate and glucose.

8. A pharmaceutical formulation according to claim 7, wherein said diluent is preferably microcrystalline cellulose and dibasic calcium phosphate dihydrate.

9. A pharmaceutical formulation according to claim 1, further comprising polyvinylprolidone polyvinylpyrrolidone (povidone) as a binding agent.

10. A pharmaceutical formulation according to claim 1, comprising magnesium stearate as a lubricant.

11. A method for preparing a controlled-release pharmaceutical formulation, said method comprising the steps of:
a) mixing together with colloidal silicone dioxide and pramipexole dihydrochloride monohydrate after sieving;
b) adding dibasic calcium phosphate dihydrate, glyceryl behenate, polymethacrylate, copovidone(polyvinylpyrolidone-vinyl acetate copolymer), microcrystalline cellulose into this powder mixture and mixing the resulting mixture;
c) adding magnesium stearate to the mixture after sieving and mixing the resultant mixture for a short period of time; and
d) compressing the resulting mixture into tablets, or filling this powder mixture into capsules.

12. A method for preparing a pharmaceutical formulation made in accordance with the formulation of claim 1, said method comprising the steps of:
a) mixing together with polyvinylprolidone, dibasic calcium phosphate dihydrate and pramipexole dihydrochloride monohydrate after sieving in a high-shear granulator;
b) melting glyceryl behenate and spraying this melt into the powder mixture prepared, thereby yielding wet granules and then cooling and sieving the wet granules;
c) adding polymethacrylate, microcrystalline cellulose and colloidal silicone dioxide into this powder mixture and mixing the resulting mixture;
d) adding magnesium stearate into this mixture and blending the resulting mixture until a uniform powder mixture is obtained; and
e) compressing the blended mixture in order to obtain tablets, or filling the powder mixture into capsules.

13. A method for preparing a pharmaceutical formulation made in accordance with the formulation of claim 1, comprising the steps of:
a) dissolving pramipexole dihydrochloride monohydrate and polyvinylpyrrolidone in water and/or alcohol to produce a pramipexole dihydrochloride monohydrate solution;
b) adding dibasic calcium phosphate dehydrate into the resulting solution while it is mixed, then it is blended in a high-shear granulator to produce wet granules;
c) adding solution of polymethacrylates into the resulting mixture;
d) sieving, and then drying the wet granules;
e) adding glyceryl behenate, microcrystalline cellulose and then colloidal silicone dioxide into the resulting powder;
f) adding magnesium stearat into this mixture and blending the resulting mixture until a uniform powder mixture is obtained; and
g) compressing the blended mixture in order to obtain tablets, or filling the powder mixture into capsules.

14. A method for preparing a pharmaceutical formulation made in accordance with the formulation of claim 1, comprising the steps of:
a) dissolving pramipexole dihydrochloride monohydrate and polyvinylpyrrolidone in water and/or alcohol to produce a pramipexole dihydrochloride monohydrate solution;
b) while the resulting solution is mixed, adding dibasic calcium phosphate dihydrate, microcrystalline cellulose, and glyceryl behenate into this solution and mixing the same, thereafter blending it in a high-shear granulator to produce granules;
c) adding solution of polymethacrylates into the resulting mixture;
d) sieving, and then drying the wet granules, thereafter disintegrating the dry granules;
e) adding colloidal silicone dioxide and mixing the resultant mixture;
f) adding magnesium stearate into this mixture and blending the resulting mixture until a uniform mixture is obtained; and
g) compressing the blended mixture in order to obtain tablets, or filling the powder mixture into capsules.

15. A pharmaceutical formulation, said formulation consisting of:
a) pramipexole dihydrochloride monohydrate at 0.05 to 5% by weight as an active agent;
b) dibasic calcium phosphate dihydrate at 5 to 90% by weight as an excipient;
c) glyceryl behenate and polymethacrylates at 5 to 90% by weight;
d) polyvinylprolidone at 0.1 to 30% by weight;
e) microcrystalline cellulose at 5 to 90% by weight;
f) silicone dioxide at 0.1 to 10% by weight; and
g) magnesium stearate at 0.1 to 10% by weight, each of c)-g) added as controlled release agents to said formulation.

16. A pharmaceutical formulation according to claim 1, for use in the
prevention or treatment of Parkinson's disease and restless leg syndrome in mammalians, particularly in humans.
